(19) United States
(12) Patent Application Publication
(10) Pub. No.: US 2014/0268037 A1
(43) Pub. Date: Sep. 18, 2014

(54) INTELLIGENT HEADREST AND OPHTHALMIC EXAMINATION AND DATA MANAGEMENT SYSTEM

(71) Applicant: Neuroptics, Inc., Irvine, CA (US)

(72) Inventor: Kamran Siminou, Newport Coast, CA (US)

(21) Appl. No.: 14/213,819

(22) Filed: Mar. 14, 2014

Related U.S. Application Data
(60) Provisional application No. 61/793,920, filed on Mar. 15, 2013.
(60) Provisional application No. 61/801,756, filed on Mar. 15, 2013.

(51) Int. Cl. A61B 3/11; A61B 3/00

(52) U.S. Cl. CPC A61B 3/11; A61B 3/0083
USPC 351/205; 351/245

(57) ABSTRACT

An automated ophthalmic diagnostic system includes a portable, hand-held ophthalmic instrument and a headrest detachably attachable to the ophthalmic instrument. The ophthalmic instrument has an RFID reader, a microprocessor, and a display. The headrest has a read/write RFID tag that stores patient identification information and sequential ophthalmic data and can transmit that information to the ophthalmic instrument through near-field communication. The microprocessor has a program that analyzes the sequential ophthalmic data and provides an output on the display, wherein a trend within the ophthalmic data is visually identifiable in the output.

The present invention claims priority from U.S. Provisional Application Ser. No. 61/793,920, filed Mar. 15, 2013 and U.S. Provisional Application Ser. No. 61/801,756, the entirety of each are incorporated herein by reference.

### BACKGROUND

This disclosure relates to instruments, systems, and methods of monitoring the eyes of subjects in a clinical or medical setting, such as in a doctor's office, medical clinic, hospital, or mobile clinical unit, such as a roaming clinic on wheels.

Each year there are nearly 1.4 million cases of traumatic brain injury resulting in 52,000 deaths in the United States. It is recognized that intensive bedside neuromonitoring is critical in pre-venting secondary ischemic and hypoxic injury common to patients with traumatic_brain injury in the days following trauma. Cecil, S et al. (2011) Crit. Care Nurse V. 31, No. 2, PP. 25-36. Multimodal monitoring of patients with traumatic brain injury is becoming more common to detect signs of secondary neurological damage. Id. at page 25. However, the onset and extent of secondary injury are not easily detected. Intensive monitoring is therefore very important to improving the prognosis of such patients. Signs of secondary neurological damage include brain swelling, somnolence, abnormal motor function, and pupillary changes. Id. Thus, consistent examination of such patients with a pupilometer or other ophthalmic instrument to detect changes in eye function have become more common in the clinical setting.

What has also emerged as an important factor in monitoring a brain injury patient's well-being is recognizing trends in pupillary response to stimulus. One tragic example of a failure to account for such a change is described in the case of 24 year old patient who died as a result of confusion in detecting and reporting a change in the pupillary response of the patient. The patient's pupil became dilated and non-responsive as a result of a brain rupture. Immediate action should have been taken to operate on the patient. But no action was taken, because there was confusion as to whether the nurse who checked the patient's pupils reported that the pupillary response was sluggish versus non-responsive. As a result of this confusion, no immediate action was taken to operate on the patient, and the patient died. This example illustrates the need for automated pupilometry and other ophthalmic systems that can minimize the risk of such tragic results from occurring in the future by automating the detection of the pupillary response and providing automatic trends that are highlighted and that trigger alarm signals when serious conditions are detected.

Many different kinds of ophthalmic diagnostic instruments are used in the field to gather information about the health or condition of a patient's eyes or the health or condition of a patient in general, particularly the health or condition of a patient's brain or nervous system. Light, and easy to carry hand-held devices are gaining favor among medical practitioners for their ease of use and their mobility to move from hospital room to hospital room. Such devices are used by medical practitioners to check on the health of their patients, especially patients who have suffered traumatic brain injury.

One such device is a hand-held pupilometer, which can gather information about a patient's pupillary response to a light stimulus or other stimulus. U.S. Pat. No. 8,235,526, which is incorporated herein by reference in its entirety, describes such a pupilometer. Hand-held pupilometers can be carried by doctors or nurses from station to station or from hospital room to hospital room to check on many different patients over a short period of time. Such devices are generally not inexpensive, and a medical facility, such as a hospital or a medical clinic, may only have one or a small hand-full of such devices for use by all of the medical staff within the entire medical facility.

Such pupilometers are typically used as follows. One or a small hand-full of pupilometers are located throughout a medical facility. They are usually positioned within a cradle when not in use. The hospital staff will check a pupilometer out when needed. The user takes the pupilometer it to the hospital room where the patient is located and checks the patient's pupillary response. The response is recorded by hand on the patient's medical chart or sometimes saved within the memory of the pupilometer. The information is critical, as explained above.

However, there is as of yet no convenient data management system that can be used to track the condition over time of each patient among a multitude of patients throughout a hospital or other medical facility where dozens or even hundreds of patients are treated every day for injuries or diseases that benefit from monitoring changes in pupillary or other ocular response.

As highlighted above, ophthalmic data obtained from a patient can be vital to the well-being and mortality of the patient. Particularly, recognizing a trend within the data gathered from a patient can lead to the implementation of life-saving measures, or conversely, failure to recognize a trend can result in various degrees of injury and harm to the patient.

Thus, what is needed is a convenient, easy-to-use, and automated system that can be used with hand-held pupilometers in the field to obtain, store, recall, transmit, and access from multiple different locations or devices a patient's ophthalmic data. What's also needed is a system that can easily and quickly provide trends or alarms to medical practitioners with respect to such ophthalmic data, such as providing a trend relating to pupillary response changes over time.

### SUMMARY

In accordance with one embodiment, described is an automated ophthalmic diagnostic system that includes a portable, hand-held ophthalmic instrument and a headrest detachably attachable to the ophthalmic instrument. The ophthalmic instrument has a an RFID reader, a microprocessor, and a display. The headrest has a read/write RFID tag that stores patient identification information and sequential ophthalmic data and can transmit that information to the ophthalmic instrument through near-field communication. The microprocessor has a program that analyzes the sequential ophthalmic data and provides an output on the display, wherein a trend within the ophthalmic data is visually identifiable in the output.

In accordance with another embodiment, a headrest is described. The headrest is attachable to a hand-held ophthalmic instrument. The headrest includes an instrument interface with a proximal end and a distal end. The instrument interface has a release mechanism on its proximal end to disengage the headrest from an ophthalmic instrument. It also includes a radio-frequency identification (RFID) tag that can receive and transmit ophthalmic data. The instrument interface also has an orbital rest extending distally from its distal end. The headrest also has a facial interface with a proximal end and a distal end, wherein the proximal end of the facial interface is attached to the distal end of the instrument interface, and the distal end has an eyelid grip.

In accordance with another embodiment, a computer program product is described. It is embodied in a non-transitory computer-readable storage medium and has computer-executable instructions recorded on said storage medium for performing a method having the following steps: receiving two or more sets of ophthalmic data from an RFID tag associated with an ophthalmic instrument headrest; processing said ophthalmic data; and generating an output, wherein a trend within the ophthalmic data is visually identifiable in the output.

In another embodiment, a computerized method for diagnosing a human subject is described. The computerized method includes the following steps: receiving two or more sets of ophthalmic data from an RFID tag associated with an ophthalmic instrument headrest; processing said ophthalmic data; and generating an output, wherein a trend associated with the ophthalmic data is visually identifiable in the output.

In another embodiment, a pupilometer is described. The pupilometer has an iris scanner, a display, a microprocessor with a memory, a computer program associated with the microprocessor that compares two sets of iris image data and issues an error message on the display if the two sets of iris image data do not match, and a camera that can detect and image a pupil.

In another embodiment, another pupilometer is disclosed. The pupilometer has a display, a microprocessor with a memory, a camera that can detect and image a pupil, and a computer program associated with the microprocessor. The program can receive two or more different sets of pupilary data from an RFID tag on a headrest attached to the pupilometer, process said two or more different sets of pupilary data, and generate an output on the display, wherein if there is a trend within the pupilary data the trend is visually identifiable in the output on the display.

### BRIEF DESCRIPTION OF THE DRAWINGS

FIG. 1 is a front-oriented perspective view of a headrest in accordance with one embodiment.

FIG. 2 is a rear-oriented perspective view of the headrest depicted in FIG. 1.

FIG. 3 is a view of the headrest taken from the rear or proximal end of the headrest depicted FIG. 1.

FIG. 4 is a side view of the right side of the headrest depicted in FIG. 1.

FIG. 5 is a top view of the headrest depicted in FIG. 1.

FIG. 6 is a perspective rear-oriented perspective view of a headrest and pupilometer assembly.

FIG. 7 is a top view of the headrest and pupilometer assembly depicted in FIG. 6 in accordance with one embodiment.

FIG. 8 is a side view of the headrest and pupilometer assembly depicted in FIG. 6.

FIG. 9 is a front-oriented perspective view of the headrest and pupilometer assembly depicted in FIG. 6.

FIG. 10 is a side view of the right side of a headrest and eyecup assembly in accordance with one embodiment.

FIG. 11 is a view of the headrest and eyecup assembly depicted in FIG. 10 taken from the rear or proximal end of the assembly.

FIG. 12 is a view of the headrest and eyecup assembly depicted in FIG. 10 taken from the front or distal end of the assembly.

FIG. 13 is a top view of the headrest and eyecup assembly depicted in FIG. 10.

FIG. 14 is a bottom view of the headrest and eyecup assembly depicted in FIG. 10.

FIG. 15 is a front-oriented perspective view of the headrest and eyecup assembly depicted in FIG. 10.

FIG. 16 is a diagram of a system architecture of an ophthalmic diagnostic and data management system in accordance with one embodiment.

### DETAILED DESCRIPTION

As set forth above, there is a need for ophthalmic instruments and ophthalmic data management systems that automate the way that ophthalmic data from patients is collected, managed and used to guide patient care. This is particularly true with respect to the care of patients who have undergone neurological or brain trauma or have undergone a surgical procedure that requires consistent monitoring of their pupils to ensure that there is no neurological or brain worsening of the patient. Pupilometers exist, but what is needed is a way of ensuring the integrity of the data obtained from the patient, a way of automating the identification of trends in the patient's neurological status, and a way of signaling when the patient's condition is serious and requires immediate attention.

Provided here are devices, systems, and programs that meet these needs. FIG. 16 is a diagram that illustrates an automated system of patient care. The system involves obtaining ophthalmic data from a patient repeatedly over time, automatically analyzing that data, automatically generating trends contained within the data that can be visualized, automatically identifying a trend that indicates a serious medical condition, and automatically issuing an alarm signal to indicate that the patient is suffering from a serious medical condition that requires immediate attention. As shown in FIG. 16, the system includes the following components: (1) an ophthalmic instrument 200, such as a pupilometer; (2) a headrest 100 attachable to the ophthalmic instrument 200; (3) a barcode scanner 500; (4) a barcode bracelet or sticker 550 that contains the patient's unique identification number or other identification information; (5) a portable computer 600, such as a laptop, tablet, or smart mobile telephone; an emergency medical records (“EMR”) system and database 700; and a cloud database 800 of patient medical information. The system architecture and function will be described in relation to the gathering and use of pupilary information from patients using a pupilometer 200, as the system is particularly suited for that activity. However, the system can be applied to other ophthalmic information gathered by other automated hand-held ophthalmic instruments.

A hospital or medical facility may only have one or handful of pupilometers 200 throughout the facility. That pupilometer 200 is typically needed to manage the care of a large number of patients who require consistent monitoring of their pupils. The condition of the pupils (i.e., size, shape, dynamic response to stimulus, such as light, or a comparison between the patient's two pupils in terms of size, shape or dynamic response to stimulus) can be indicative of the patient's neurological status or condition, particularly the condition of the patient's brain. This is discussed in U.S. Pat. Nos. 7,967,442, 8,235,526, 8,393,734 and U.S. application Ser. No. 12/436,469, all of which are incorporated herein by reference in their entireties. Thus, for example, with patients who have suffered brain trauma, are comatose, have a brain tumor or who have undergone a surgical procedure and who may be unconscious or semi-conscious, standard patient care can involve monitoring the patient's pupils. The system described herein can be used to monitor such patients' pupils.

Each patient in a hospital or medical clinic is assigned a unique patient identification number. All of the patient's personal, medical, and treatment information are associated with that ID number. No two patients have the same ID number. Each patient is given a wristband or other article they carry that has a barcode 550 with the ID number. The same barcode 550 is printed onto stickers and can be affixed to the patient's drug prescriptions, medical devices, and other articles. A sticker with the barcode can also be affixed to a pupilometer headrest 100 and assigned to the patient. The headrest 100 is used whenever the nurse, doctor or other healthcare provider takes pupillary measurements of the patient's pupils.

The healthcare provider arrives at the patient's bedside to take the pupillary measurements. The healthcare provider uses a pupilometer 200 to takes those measurements. Before the measurements can be taken for the first time, the patient's unique ID number must be received by the pupilometer 200. This can be accomplished in one of three ways. The first is by inputting the patient's ID number manually into the pupilometer 200 using either a keypad or touchscreen on the pupilometer 200 or using voice recognition if the pupilometer 200 has voice recognition capability. The second is by using a barcode scanner 500 to scan the patient's unique barcode 550. Once the barcode scanner 500 has obtained the patient's unique patient ID from his or her barcode 550, the scanner 500 transmits that data to the pupilometer 200. The transmission of that data can be either through a wired cable connection between the scanner 500 and the pupilometer 200, or through a wireless connection, such as blue tooth, radio frequency, near-field communication (NFC), or other wireless communication protocols.

The headrest must also be assigned that unique patient ID number before it can be used for the first time, and this can happen in either of three ways. The first is that the scanner 500 has a built in RFID read and can transmit the patient ID data to the headrest 100 RFID tag 150 through a wireless RFID signal. The RFID reader has the standard electronics needed for the scanner to transmit and record data onto the RFID tag 150 of the headrest 100. The transmission of that patient ID data can be through a wireless connection, such as blue tooth, radio frequency, near-field communication (NFC), or other wireless communication protocols that are typically used between an RFID reader and an RFID tag. The second way is for the scanner 500 to first transmit the patient ID data to a portable computing device 600, which can be used to transmit that data to the RFID tag 150 on the headrest 100. The portable computing device 600 can have a built in RFID read and can transmit the patient ID data to the headrest 100 RFID tag 150 through a wireless RFID signal. The RFID reader of the portable computing device 600 can have the standard electronics needed for it to transmit and record data onto the RFID tag 150 of the headrest 100. The transmission of that patient ID data can be through a wireless connection, such as blue tooth, radio frequency, near-field communication (NFC), or other wireless communication protocols that are typically used between an RFID reader and an RFID tag.

The third way to transmit the patient ID to the headrest 100 is through the pupilometer as described next. Once the pupilometer has the patient's unique ID number, the healthcare provider takes the clean, unused headrest 100 that is assigned to the patient and attaches it to the pupilometer 200. This headrest can be kept at the patient's bedside and can also have a barcode sticker or other identifying indicia, which associates it uniquely with that patient. The pupilometer 200 has an RFID reader (a radio-frequency transmitter-receiver) that can send a signal to the RFID tag 150 on the pupilometer and can receive and read its response. Thus, the pupilometer is capable of communicating with the RFID tag 150 on the headrest 100 through a radio-frequency or other NFC or short-range wireless signal.

But before it can communicate with the headrest 100, the pupilometer 200 has to first detect the presence of the headrest 100. The pupilometer 200 detects the presence of the headrest 100 in one of two ways. The headrest 100 can have a magnet 180 imbedded in it (as shown in FIG. 2), and the pupilometer will have a mechanism to detect the magnet 180, such as a polar opposite magnet or metal that is wired to a processor in the pupilometer. Alternatively, the pupilometer 200 constantly transmits a signal and searches for a response that contains either (1) a patient ID or (2) another signal associated only with headrests 100. The strength of the RFID signal on the pupilometer 200 is such that the headrest 100 must either be coupled to the pupilometer or be within inches of the pupilometer 200 at most in order for the two devices to be able to communicate with one another through the RFID signal.

Once the pupilometer 200 detects the presence or attachment of the headrest 100, the pupilometer 200 searches for a patient ID number on the RFID tag 150. If there isn't one, the pupilometer 200 will write the patient's ID number onto the RFID tag 150, where it gets written into the memory of the RFID tag 150. Once the ID number is written into the memory of the RFID tag 150, the pupilometer 200 will never again write a new ID number into the memory of that particular RFID tag 150. Now that headrest 100 is associated uniquely with the patient identified by the unique patient ID, and that headrest 100 can never again be associated with a different patient.

Now that the patient ID has been entered into the pupilometer 200 and transmitted to the headrest 100, the healthcare provider is ready to take the patient's first pupil measurements. With the headrest 100 attached to the pupilometer 200, the pupilometer 200 is placed in front of the patient's first eye, the eyelid is opened, and the patient's pupil is brought into the field of view of the pupilometer 200 camera and an image of the pupil or a short video of the pupil is recorded by the pupilometer. Pupilometer's such as the ones described in U.S. Pat. Nos. 7,967,442, 8,235,526, 8,393,734 and U.S. application Ser. No. 12/436,469, can be used in the present system in order to capture pupillary data from the patient. Such data can be static pupil data, such as the size or shape of the pupil. The camera of the pupilometer 200 can also record the pupil's dynamic response to the stimulus. All of that data is saved in the memory of the pupilometer 200 and is associated with the patient ID. The pupilometer 200 can then be used to record the same information from the patient's other pupil and save that data as well. Next, all of that pupillary data is transmitted by the RFID reader to the RFID tag 150 on the headrest 100 and written into the memory of the RFID tag 150. The headrest 100 is then removed from the pupilometer 200 and placed back by the bedside of the patient, and the healthcare provider can move on to the next patient and repeat the above steps. Meanwhile, all of the pupillary data that was just obtained from the patient is now saved in the memory of the RFID tag 150 and associated with that particular patient.

For added safety controls, the pupilometer 200 can include a biometric reader that scans the iris of the patient's eye. The biometric reader on the pupilometer 200 can be used to scan one or both of the patient's iris' and associate a digital representation of that scan with the patient's unique ID. The pupilometer 200 can transmit that iris data also to the headrest 100 where it can be stored in the memory of the RFID tag 150. Each time the pupilometer 200 is used to take measurements of a patient's pupils, the pupilometer 200 will first scan the iris of the patient and compare it to the iris data contained in the RFID tag 150 of the headrest 100. If the iris data does not match, the pupilometer 200 will show an error message or issue a warning indicating that the iris data in the headrest 100 does not match the iris data from that patient. This indicates that the headrest 100 belongs to a different patient and should not be used to take the pupilary measurements of this patient. The purpose of this safety control is to eliminate the risk of comingling pupilary measurements of different patients, because the headrest 100 will only be used for a single patient, and all of the pupilary data from that headrest will come from that one patient without the risk that it contains data from another patient.

The pupilometer 200 can be in wireless communication with the electronic medical records (“EMR”) system database 700 of the hospital or clinic, and can transmit the pupillary information to the EMR 700. It can also be used to retrieve information from the EMR 700 if necessary.

Later, the doctor, nurse or other healthcare provider can return to the same patient to take another pupillary measurement. At that time, the healthcare provider will take the headrest 100 from the bedside and attach it to the pupilometer 200. If the hospital or clinic has more than one pupilometer 100, then the healthcare provider may not be using the same pupilometer that was used earlier. However, the same pupilometer is not required, because all of the patient information and pupillary information associated with that patient is saved in the memory of the headrest RFID 150 at the bedside of the patient. The pupilometer 100 will recognize when the headrest 200 is attached and will read the patient ID number off of the RFID tag 150. It will also conduct the iris matching protocol to confirm that the headrest 100 belongs to the patient whose measurements are now going to be taken. The pupilometer 200 will also retrieve and read the pupillary response data that was written earlier into the memory of the RFID tag 150. All of that pupilary data is now transmitted to the pupilometer 200. The pupilometer 200 is again used to repeat the steps described above in obtaining pupillary information from one or both of the patient's eyes. That information is again saved into the memory of the pupilometer 200, and again subsequently transmitted and written into the memory of the RFID tag 150 of the headrest 100. It can also be transmitted to the EMR 700 again.

Now the pupilometer 200 and the RFID tag 150 both have two sets of pupillary data. The number of data sets grows with each time that the pupilometer 200 and headrest 100 are used as part of the patient care to monitor the condition of the patient. The pupilometer 200 has pupillary analysis software that can do all of the following: (1) display the pupillary data on the display of the pupilometer; (2) analyze all of the data sets and convert the pupillary data into a visual aid that can be used to identify a trend; (3) identify a trend within the pupillary data that indicates a serious neurological abnormality or problem; and (4) generate a visual or sound signal upon identifying a trend within the pupillary data that indicates the patient is suffering from a serious neurological or other medical condition. With respect to item 2 above, such a visual aid can be one or more graphs or charts that show one or more aspects of the pupillary data. For example, the chart can track the pupil's maximum size at rest, or the pupil's maximum constriction in response to a stimulus. The chart can include all of the data points for one or more of those or other parameters. The healthcare provider can determine with a quick review of the chart whether there is cause for concern with respect to a patient's neurological status or medical condition. If the patient is suffering from a serious neurological or other medical condition, the pupilometer 200 can signal an alarm visually or generating a sound that is emitted by a speaker on the pupilometer 200. For example, if the patient is suffering from a serious brain injury and the patient's brain condition is deteriorating, a trend in one or both (or a comparison of the two) of the patient's pupils will be detected by the pupilometer software and the software will generate an alarm that will signal that the patient's neurological or other medical condition is serious and that the patient requires immediate medical attention. This can be an earlier warning signal to alert medical staff to an impending emergency so that they can take action earlier than they might have otherwise.

The pupillary data analysis software on the pupilometer 200 can also be used elsewhere. For example, the software can be loaded onto a portable computing device 600, such as a laptop computer, tablet computer, smart phone or the like. The portable computing device 600 can be taken from patient to patient throughout the hospital or clinic and gather pupillary data from the headrests 100 at the patients' bedside. Like the pupilometer 200, the portable computing device 600 also has an RFID reader and can receive, read and record information it receives from the RFID tag 150. When brought into range of the RFID tag 150 of the headreset 100, the portable computing device 600 can retrieve all of the data stored in the memory of the RFID tag 150, and can process it using the same software that the pupilometer 200 has. Thus, the portable computing device 600 can also display the pupillary data on its display; (2) analyze all of the data sets and convert the pupillary data into a visual aid that can be used to identify a trend; (3) identify a trend within the pupillary data that indicates a serious neurological abnormality or problem; and (4) generate a visual or sound signal upon identifying a trend within the pupillary data that indicates the patient is suffering from a serious neurological or other medical condition.

The portable computing device 600 can transmit wirelessly (or through a wired LAN or other connection) to the EMR 700 all of the pupillary data it has gathered from the various headrests 100 from which it has acquired pupillary data. This is an easy and convenient way to obtain pupillary data from all of the patients whose pupils are being monitored and transmit that data to the EMR 700. The portable computing device 600 can also communicate through an Internet connection with a cloud-based database or processing center 800, and can send and retrieve data from that center 800.

In one embodiment of a pupilometer 200 as described above, the pupilometer 200 has a built in biometric iris scanner (sometimes called iris reader). The pupilometer can also have a built-in infra-red light to illuminate the eye during a biometric iris scan. The pupilometer 200 can thus be used to scan the iris of a patient's eye. The iris image data is saved into the memory of the pupilometer 200. The pupilometer 200 can transmit that iris image data to the RFID tag 150 of the headrest 100 where it is written into the memory of the RFID tag 150. The pupilometer 200 has iris image data comparison software. The software performs the following function and steps. Before the pupilometer measures the patient's pupils, the pupilometer will perform an iris scan of the patient. The pupilometer 200 will save that data in its memory. The pupilometer 200 will seek out the iris image data in the RFID tag 150 of the headrest 100 that is attached to the pupilometer 200 and will retrieve that data from the RFID tag 150. The pupilometer 200 will then compare the iris image data it just obtained from the patient to the iris image data it just retrieved from the headrest 100 RFID tag 150. If the pupilometer 200 does not detect a match between the two sets of data, it will display an error message on its display, or a message indicating that there was no match, or a message indicating that the headrest does not belong to this patient.

The present system as depicted in FIG. 16, provides a great improvement in patient care for patients who require consistent monitoring of their pupils. First, it reduces the risk of data contamination or comingling among different patients. Second, it makes trends in data more reliable, because of the reduced risk of data contamination among patients. Third, it makes the sharing and management of pupillary data easier and more convenient. Fourth, it provides an objective warning signal to the healthcare provider that a patient's neurological or other medical condition is serious and that he or she requires immediate medical attention. The present system reduces or eliminates the subjectivity associated with analyzing pupilary data or the response of a pupil to a stimulus. A healthcare provider no longer has to guess as to whether or not the patient's neurological condition is stable, worsening or dire. The software will give the provider a visual depiction of any trends contained within the patient's pupilary data, and a warning signal if it detects a trend that indicates a serious neurological or medical condition.

The pupilometers 200 described herein contain associated control units and software for analyzing the activity of a patient's pupil(s) and providing various outputs as described above. Such outputs or signals can be indicative of various neurological disorders or neurological conditions, including those associated with optic nerve disease, brain damage, brain tumors, brain worsening, stroke, or other serious medical conditions that require immediate medical attention.

Other ophthalmic instruments can be adapted to the system depicted in FIG. 16. Instruments, such as hand-held and/or portable tonometers, retinascopes, and opthalmoscopes may be used if adapted to include microprocessors and software such as that described herein. Like the pupilometer 200 described above, such instruments can be used to analyze, manage, and depict data associated with the state of the patient's eye(s).

An important aspect of the system described above is a low-cost, disposable headrest that can read, write and transmit data and that can be attached to handheld portable ophthalmic instruments, such as pupilometers. FIGS. 1-15 depict such a headrest 100 and variations of that headrest.

Turning now to FIGS. 1-5, a headrest 100 is provided that can be attached to a pupilometer 200, such as the one shown in FIG. 6. The headrest 100 can be made of any rigid material, including plastic, metal, stainless steel, titanium, and the like. The headrest 100 has a proximal end 110 and a distal end 120. The proximal end 110 is designed to be attached to a pupilometer. An orbital rest projects distally from the distal end 120 of the headrest 100. The orbital rest has an arm 130 extending distally from the distal end 110, and a face stop 140 at the distal end of the arm 130. The face stop 140 extends in a downward direction from the distal end of the arm 130. The proximal end of the arm 130 is attached to the distal end 120 of the headrest 100. The face stop 140 is rigid, but it can have a soft padding 142 affixed to its distal side. The soft padding 142 is the interface and contact point with a subject's face. The arm 130 can be between 1 cm and 10 cm long, and in various embodiments, it is about 1 cm, 2 cm, 3 cm, 4 cm, 5 cm, 6 cm, 7 cm, 8 cm, 9 cm, or 10 cm long. The orbital rest is used to stabilize the headrest 100 against a subject's face before taking a measurement of the subject's pupils. This reduces the amount of movement of the pupilometer when the pupil measurements are taken, and that allows for more accurate measurements and data.

The proximal end of the headrest 110 is designed to fit snugly around the distal end of a lens neck 250 of a pupilometer 200 (see FIGS. 6-9). The headrest 100 is generally cylindrical in shape as shown in FIGS. 1-5, although it can be adapted to be square or rectangular if the lens neck of the pupilometer is square or rectangular. The proximal end 110 of the headrest 100 has five sections, each separated or interrupted by a gap. The gaps allow the sections to flex slightly with respect to one another so that the proximal end 110 of the headrest can expand slightly to fit snugly over the lens neck 250 of the pupilometer 200. The headrest 100 can be made of a rigid material, such as plastic, that can flex slightly without cracking or chipping.

The top section 175 of the headrest 100 is the largest uninterrupted section. The top section 175 has an RFID tag 150 affixed to its top surface. The top section 175 of the headrest 100 fits over the top of the distal end of the lens neck 250 of the pupilometer (see FIGS. 6-9). Top section 175 is book-ended by gaps 61 and 62, which separate the top section 175 from release tabs 165a and 165b. Lower sections 170a and 170b form the remaining two sections of the proximal end 110 of the headrest 100. Those two sections are separated from one another by gap 24. Section 170a is separated from release tab 165a by gap 65, and section 170b is separated from release tab 165b by gap 63.

Release tabs 165a and 165b are substantially opposite one another along the radius of the headrest 100. Release tabs 165a and 165b are substantially rectangular in shape and smaller than any of the other sections of the headrest 100. The distal end of each of the release tabs 165a and 165b is formed integrally with the distal 120 end of the headrest 100. The ability of the release tabs 165a and 165b to flex radially outward away in a direction away from one is proportional to the length of the release tabs (i.e., the distance from the proximal end of the release tab to the distal end of the release tab). The longer that distance, the more release tab can flex outward, and the shorter that distance the less the release tab can flex outward. The proximal end of each of the release tabs 165a and 165b has an indentation 168a and 168b respectively. Each of indentations 168a and 168b fits over and mates with a corresponding protuberance (not shown) on the outer wall of the distal end of the pupilometer 200 lens neck 250. When the indentations mate with their respective protuberance, the headrest 100 is locked to the pupilometer 200. Alternatively, the release tabs 165a and 165b can have protuberances instead of the indentations 168a and 168b and can mate with indentations in the outer wall of the distal end of the pupilometer 200 lens neck 250.

To release the headrest 100 from the pupilometer 200, the headrest 100 has a pair of opposing pinchers 160a and 160b. The pinchers 160a and 160b are each connected to release tabs 165a and 165b respectively through connectors 167a and 167b respectively. Squeezing the two pinchers 160a and 160b together causes the proximal ends of the release tabs 165a and 165b to bend away from one another radially outwardly from the headrest 100. When the release tabs 165a and 165b bend outward, the indentations 168a and 168b become released from the protuberances on the pupilometer 200 lens neck 250, thus releasing the headrest 100 from the pupilometer 200.

The headrest 100 can also have a magnet contained within magnet housing 180. The magnet can be detected by the pupilometer 200 so that when the headrest 100 is connected to the pupilometer 200, the pupilometer 200 recognizes that the headrest 100 is attached to it. The pupilometer 200 contain electronics to detect the magnet and logics to switch to assembled mode once the magnet is detected.

As shown in FIG. 6, the pupilometer has a handle 220, a display 210 and a control panel 230 to control the operation of the pupilometer 200. The headrest 100 can be coupled to the pupilometer 200 by squeezing the pinchers 160a and 160b, and sliding the proximal end of the headrest 110 past the lens 260 of the pupilometer and over the lens neck 250 of the pupilometer 200 until the protuberances (not shown) on opposite side of the lens neck 250 snap into the indentations 168a and 168b of the headrest. Once the headrest 100 is snapped onto the pupilometer 200 lens neck 250, the pupilometer 200 detects the magnet in the magnet chamber 180. After the pupilometer 200 detects the headrest 100, it can go into coupled mode.

In coupled mode, the pupilometer 200 seeks out information from the RFID tag 150 on the headrest 100. The pupilometer has an RFID reader and can send a wireless signal to the RFID tag 150 to do any of the following: power the RFID tag 150 if it is not self-powered; seek patient ID data from the RFID tag 150; seek pupilary data from the RFID tag 150; transmit patient ID data to the RFID tag 150; or transmit pupilary data to the RFID tag 150.

The RFID tag 150 can be a passive tag, an active tag or a battery assisted passive tag. The RFID tag 150 can either be read-only, or may be read-write, where data can be written into the tag by the system user. In one embodiment, the RFID tag 150 has an on-board memory where it can receive and store data. The RFID tag 150 can also transmit data. Both the reception and transmission of data to and from the RFID tag 150 is accomplished through standard wireless transmission protocols used by RFID systems, including radio-frequency transmission or other near-field (“NFC”) wireless protocols. The RFID tag 150 includes a small RF transmitter and receiver. An RFID reader, such as one built into the pupilometer 200, transmits an encoded radio signal to interrogate the RFID tag 150. The RFID tag 150 receives the message and responds with its identification information. This may be only a unique tag serial number, or may be product-related information such as a stock number, lot or batch number, production date, or other specific information. It can also include patient information or pupilary data that was previously stored in the memory of the RFID tag 150. The RFID tag 150 contains at least two parts: (1) an integrated circuit for storing and processing information, modulating and demodulating an RF signal, collecting DC power from the incident reader signal, and other specialized functions; and (2) an antenna for receiving and transmitting the signal.

FIGS. 10-15 show the headrest of FIGS. 1-5 with an additional component releasably or permanently attached to the headrest 100. That component is facial interface 400. Facial interface 400 is essentially an flexible rubberized eyecup that can rest against the subject's face around the eye of the subject. The benefit of using an eyecup is to limit the amount of ambient light that reaches the eye during measurement of the pupils.

The facial interface 400 has a proximal end 410 and a distal end 420. The proximal end 410 is coupled to the distal end 120 of the headrest 100. In one embodiment, the proximal end 410 of the eyecup is adhered to the distal end of the headrest 120 with an adhesive. In another embodiment, as shown in FIG. 11, the distal end 120 of the headrest 100 has a number of holes on its perimeter and the proximal end 410 of the facial interface 400 has a number of protuberances 470 matching the number of holes on the headrest 100 distal end 120. The protuberances 470 are flexible and slightly larger in diameter (at least on their proximal ends) than the diameter of the holes. The protuberances 470 can each be pushed through the holes of the headrest 100 distal end 120 so that the facial interface 400 becomes releasably coupled to the headrest 100 as a result of the insertion of the protuberances 470 through the holes the holes of the headrest 100 distal end 120.

Facial interface 400 has a bottom section 450 that interfaces with the bottom of a subject's eye socket just above the cheek. The facial interface also has a top section that includes finger ports 430a and 430b as well as a eyelid grip 440. Eyelid grip 440 forms a lip that is shaped and sized to interface with a human eyelid. The eyelid grip 440 is centered between the two finger ports 430a and 430b. The finger ports 430a and 430b are sized and shaped to each allow a human finger to be inserted through the port when the facial interface 400 is resting against a subject's face. Thus, when the facial interface 400 is resting against the subject's face, and particularly around the subject's eye socket, the distal end of the facial interface 400 is in continuous contact with the skin of the subject, except that the continuous contact is interrupted at the finger ports 430a and 430b. Thus, only finger ports 430a and 430b allow light to enter into the space between the subject's eye and the pupilometer 200 lens 260. The operator of the pupilometer 200 can insert his or her fingers into the finger ports 430a and 430b to assist in opening the subject's eyelid. In an alternative embodiment (not shown) the finger ports are covered with a flexible rubber or a fabric that allows for a fingers to be inserted through the finger ports while maintaining the finger ports covered when fingers are not inserted through them. Such an embodiment prevents light from entering the space between the camera of the pupilometer 200 and the patient's eye. In such an embodiment, the rubber over the finger ports is thinner and/or more flexible than the rubber that makes up the rest of the facial interface 400, or the material is a flexible fabric.

Alternatively, eyelid grip 440 can be used as follows to assist in opening the eyelid. The pupilometer operator attaches the assembly with the headrest 100 and facial interface 400 to the pupilometer as described above. Then taking the pupilometer 200 by its handle 220, the operator brings the pupilometer up to the subject's face and positions the facial interface 400 so that it surrounds the subject's eye. The operator positions the facial interface 400 so that the eyelid grip 440 rests firmly against the subject's eyelid. The operator holds the facial interface 400 against the face of the subject so that the distal end 420 of the facial interface surrounds the subject's eye socket and is in direct contact with the subject's skin. At this point the entirety of the distal end 420 of the facial interface 400 is in direct contact with the subject's face, except the finger ports 430a and 430b. The operator then lifts the entire pupilometer 200 slightly upward while pushing it gently against the patient's face. The facial interface 400 is rubberized, so when the operator pushes the pupilometer 200 upward, the patient interface 400 does not slide against the subject's skin. Instead it pulls the skin slightly upward while maintaining a grip against the skin. The eyelid grip 440 is also rubberized. Thus, instead of sliding against the eyelid, it pulls the eyelid upward, thus opening the eyelid. In addition, the eyelid grip 440 can have a foam patch that is adhered to the underside of the eyelid grip 440 for added adhesion or friction between the eyelid grip 440 and the subject's eyelid.

The significance of the eyelid grip 440 to the design of the facial interface 400 is that it allows the pupilometer 200 operator to be able to use just one hand to quickly open the subject's eyelid and take a measurement without having to manipulate the eyelid with his or her second hand. Thus, the operator can perform the measurement using just one hand, thus freeing the other hand to accomplish other tasks, such as holding the subject's head or stabilizing the subject. This is important, because pupilometers are often used to measure the pupils of subjects who are unconscious or otherwise unable to open their eyes without assistance.

While the invention is susceptible to various modifications and alternative forms, specific examples thereof have been shown by way of example in the drawings and are herein described in detail. It should be understood, however, that the invention is not to be limited to the particular forms or methods disclosed, but to the contrary, the invention is to cover all modifications, equivalents, and alternatives falling within the spirit and scope of the appended claims.

1. A headrest attachable to a hand-held ophthalmic instrument, said headrest comprising:
an ophthalmic instrument interface with a proximal end and a distal end, wherein the ophthalmic instrument interface comprises:
a release mechanism on its proximal end to disengage the headrest from an ophthalmic instrument;
a radio-frequency identification (RFID) tag that can receive and transmit ophthalmic data;
an orbital rest extending distally from the distal end of the ophthalmic instrument interface; and
a facial interface with a proximal end and a distal end, wherein the proximal end of the facial interface is attached to the distal end of the instrument interface, and the distal end comprises an eyelid grip.

2. The headrest of claims 1, wherein the release mechanism comprises a pair of compression tabs on opposite sides of the instrument.

3. The headrest of claim 1, wherein the orbital rest comprises an arm that extends distally from the distal end of the instrument interface, and a plate that extends in an inferior direction from a distal end of the arm.

4. The headrest of claim 1, wherein the facial interface comprises a flexible material and has one or more finger ports on the perimeter of its distal end.

5. The headrest of claim 4, wherein the facial interface is made of rubber.

6. The headrest of claim 1, wherein the instrument interface further comprises a magnet.

7. An automated ophthalmic diagnostic system, comprising:
a portable, hand-held ophthalmic instrument comprising an RFID reader, a microprocessor, and a display; and
a headrest detachably attachable to the ophthalmic instrument, the headrest comprising a read/write RFID tag that stores patient identification information and sequential ophthalmic data and can transmit that information to the ophthalmic instrument through near-field communication;
wherein the microprocessor comprises a program that analyzes the sequential ophthalmic data and provides an output on the display, wherein a trend within the ophthalmic data is visually identifiable in the output.

8. The automated ophthalmic diagnostic system of claim 7, wherein the program is capable of identifying a trend within the ophthalmic data that indicates a serious medical condition.

9. The automated ophthalmic diagnostic system of claim 8, wherein the ophthalmic instrument further comprises a speaker and the program generates a visual or sound signal upon identifying a trend within the ophthalmic data that indicates a serious medical condition.

10. The automated diagnostic system of claim 9, wherein the ophthalmic instrument is a pupilometer, and the serious medical condition is neurological worsening.

11. The automated diagnostic system of claim 7, further comprising a tablet computer with an RFID reader that communicates with the RFID tag of the headrest.

12. The automated diagnostic system of claim 11, wherein the tablet computer comprises a display and a program that analyzes the sequential ophthalmic data and provides an output on the display, wherein a trend within the ophthalmic data is visually identifiable in the output.

13. The automated ophthalmic diagnostic system of claim 12, wherein the program of the tablet computer is capable of identifying a trend within the ophthalmic data that indicates a serious medical condition.

14. The automated ophthalmic diagnostic system of claim 13, wherein the tablet computer further comprises a speaker and the program of the tablet computer generates a visual or sound signal upon identifying a trend within the ophthalmic data that indicates a serious medical condition.

15. The automated ophthalmic diagnostic system of claim 10, wherein the ophthalmic data is data relating to the size, shape or dynamic response of a pupil.

16. The automated ophthalmic diagnostic system of claim 10, wherein the ophthalmic data is data relating to the size, shape or dynamic response of one pupil of a subject relative to the size, shape or dynamic response of the other pupil of the same subject.

17-23. (canceled)
